(19) United States
(12) Patent Application Publication
(10) Pub. No.: US 2016/0324762 A1
(43) Pub. Date: Nov. 10, 2016

(54) DARK CIRCLE CORRECTING AND CONCEALING COMPOSITIONS

(71) Applicant: Makefield LLC, Newtown, PA (US)

(72) Inventors: Kalyan Vepuri, Newtown, PA (US); Irwin Palefsky, Fairfield, NJ (US); Russell Grandis, Paramus, NJ (US)

(21) Appl. No.: 15/146,487

(22) Filed: May 4, 2016

Related U.S. Application Data
(60) Provisional application No. 62/156,640, filed on May 4, 2015.

(51) Int. Cl. A61K 8/97; A61K 8/34; A61K 8/63; A61K 8/64; A61K 8/19; A61K 8/58; A61Q 19/00; A61K 8/891; A61K 8/86; A61Q 1/02; A61K 8/73

(52) U.S. Cl. CPC A61K 8/97; A61Q 1/02; A61K 8/345; A61K 8/63; A61K 8/64; A61K 8/735; A61K 8/34; A61K 8/585; A61Q 19/007; A61K 8/891; A61K 8/86; A61K 8/19; A61K 2800/43; A61K 2800/49; A61K 2800/524

(57) ABSTRACT

Cosmetic concealer compositions providing a natural coverage to skin imperfections and methods of use thereof are disclosed herein.

### CROSS-REFERENCE TO RELATED APPLICATION

The present application claims priority to U.S. Application No. 62/156,640, filed on May 4, 2015, by Kalyan Vepuri, Lauren Hoffman, Irwin Palessky and Russ Gandis, the disclosure of which is incorporated herein by reference in its entirety.

### FIELD OF THE INVENTION

The present invention relates to concealer compositions providing a natural coverage to skin imperfections and methods of use thereof.

### BACKGROUND OF THE INVENTION

Cosmetic compositions such as foundations and concealers have long been used to hide perceived imperfections of the skin and to improve the aesthetic appearance of a user.

Foundations are generally intended to be applied to a person's face to create an even, uniform complexion or to change the skin tone and texture and to hide pores, imperfections, and fine lines. A foundation composition is also applied to moisturize the skin, to balance the oil level of the skin, and to provide protection against the adverse effects of sunlight, wind, and other environmental factors.

Concealers are used to obtain high coverage on a particularly concerned area, and supplement the function of a foundation. Concealers are typically liquid, paste or semi-solid form compositions containing a high level of pigments having opacity and are typically used prior to applying the foundation.

One of the drawbacks for users of concealers is that these can cause an unnatural appearance at the site of application. Since concealers are used to make a certain area of the skin less conspicuous as compared to the surrounding area, there is a need for concealer that provides coverage without increasing contrast with the adjacent areas of the skin.

Another disadvantage is that most concealers merely mask problem areas. It would be highly advantageous if one could diminish darkened skin or lines associated with the area to be concealed.

It is therefore an object of the present invention to provide a composition to address the drawbacks of current concealers to provide a natural appearance.

It is still a further object of the present invention to provide a method of lightening the skin and treating unaesthetic aspects of the skin associated with ageing.

### SUMMARY OF THE INVENTION

Compositions and methods to mask and diminish dark circles, age spots, large pores, and other small blemishes visible on the skin are described herein. An exemplary composition contains a combination of natural product extracts and hyaluronic acid to lighten as well as conceal the dark areas.

In preferred embodiments, the natural product extracts are derived from albizia julibrissin, pfaffia paniculata, ptychopetalum olacoide, lilium candidum and medicago sativa. The natural product extracts reduce dark circles and under eye bags and puffiness. Further, the natural product extracts lift the fold of the upper eyelid and lessen the crow's feet wrinkles on the sides.

The natural product extracts can be obtained by well-known techniques of extraction described below and/or are commercially available.

In preferred embodiments, the composition contains pigments, emulsifiers, dermatological agents, emollients and preservatives.

Pigments are used as coloring agents which provide color to cosmetic compositions. In one embodiment, the composition contains a combination of titanium dioxide (CI177891), red iron oxide (CI177491), yellow iron oxides (CI177492), black iron oxide (CI177499), and bismuth oxychloride. Pigment particles can be surface modified with a hydrophobic coating such as triethoxycaprylsilane. In one embodiment, the combination of the pigments and hydrophobic coating is in an amount of between about 18 and 22% weight of the composition. The composition can contain one or more emulsifiers. Emulsifiers are surface active substances which promote the suspension of one liquid in another and help maintain a stable mixture, or emulsion, of oil and water. In preferred embodiments, the emulsifier is a combination of cetyl PEG, PPG-10, 1-dimethicone and cetyl dimethicone in an amount of p to about 10%, more preferably about 8.6% weight of the composition about 8.6% weight of the composition. Emollients are externally applied compounds that soften or soothe skin. In one embodiment, the emollient is cyclopentasiloxane. Preservatives can be included to prevent microbial growth in the composition. In one embodiment, the preservative is phenoxyethanol or sodium EDTA.

In a preferred embodiment, the composition contains the following combinations of ingredients: glycerin, albizia julibrissin extract, and darutoside; water, glycerin, caprylyl/capryl glucoside pfaffia paniculata root extract, ptychopetalum olacoides extract and lilium candidum flower extract; water, medicago sativa extract, and hydrolyzed lupine protein; and sodium hyaluronate, water and phenoxyethanol. In one embodiment, the combination of glycerin, albizia julibrissin extract, and darutoside is in an amount of between about 2.8 and about 3.2% by weight of the composition. In another embodiment, the combination of water, glycerin, caprylyl/capryl glucoside pfaffia paniculata root extract, ptychopetalum olacoides extract and lilium candidum flower extract is in an amount of between about 1.8 and about 2.2% by weight of the composition. In still another embodiment, the combination of water, medicago sativa extract, and hydrolyzed lupine protein is in an amount of between about 1.8 and about 2.2% by weight of the composition. In another embodiment, the combination of sodium hyaluronate, water and phenoxyethanol is in an amount of between about 1.8 and about 2.2% by weight of the composition.

The composition can be formulated as a solution.

In certain embodiments, the composition can be applied twice a day, and preferably once a day.

### BRIEF DESCRIPTION OF THE DRAWING

FIG. 1 is a bar graph showing the differences in skin color brightness of 30 subjects treated with Composition 1 over several time points.

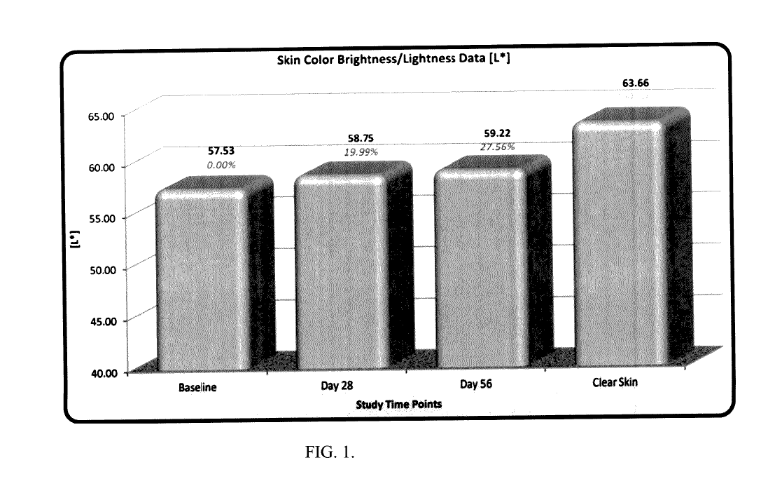

### DETAILED DESCRIPTION OF THE INVENTION

### I. Definitions

“Skin” is used herein to refer to an organ containing the epidermis, the dermis and a deep compartment, which is the hypodermis.

As used herein, the term “lightening the skin” refers generally to lightening, brightening, whitening, and/or evening of the skin tone, skin color, and/or shade of skin, and/or to the reduction in shallowness, and/or to the lightening and/or fading of hyperpigmented marks and/or lesions including, but not limited to, pigmented spots, melanin spots, age spots, sun spots, senile lentigos, freckles, lentigos simplex, pigmented solar keratosis, seborrhoeic keratosis, melasma, acne marks, post-inflammatory hyperpigmentation, lentigines, ephelides, combinations of two or more thereof and the like.

“Lightening the skin” also refers to increased skin radiance, glow, translucency and/or luminescence and/or obtaining a more radiant, glowing, translucent or luminous skin tone appearance or a less yellow or sallow skin tone.

The term “signs of ageing of the skin” means any change in the outer appearance of the skin or in its texture due to chronological or photo-induced ageing, for instance wrinkles, fine lines, wizened skin, flaccid skin, thinned skin and lack of elasticity or tonus of the skin.

“Extract”, as used herein, refers an extract from a natural product that can be obtained by well-known techniques of extraction. By way of examples, the techniques that can be used include, but not limited to, steeping, simple decoction, lixiviation, extraction under reflux, supercritical fluid extraction, extraction by means of ultrasound or microwaves, or using countercurrent technology. Extraction solvents include, but are limited to, water, propylene glycol, butylene glycol, glycerin, PEG-6 caprylic/capric glycerides, polyethylene glycol, methylic and/or ethylic diglycol ethers, cyclic polyols, ethoxylated or propoxylated diglycols, alcohols (methanol, ethanol, propanol, butanol), or any mixture thereof.

### II. Compositions

The disclosed composition includes at least one of julibrissin extract, darutoside, pfaffia paniculata root extract, ptychopetalum olacoides extract, lilium candidum extract, medicago sativa extract, and hydrolyzed lupine protein, preferably, two, three, four, five, six of these extracts and most preferably, all seven. The extract can be a root, bark, leaf or flower extract. The pfaffia paniculata is preferably a caprylyl/capryl glucoside pfaffia paniculata root extract. The lilium candidum extract is preferably a flower extract. The Ptychopetalum olacoidesis extract is preferably bark and/root.

In one embodiment, the combination of glycerin, albizia julibrissin extract, and darutoside is in an amount of 2.8-3.2% by weight of the composition, for example, 2.8, 2.9, 3.0, 3.1, and 3.2%. In another embodiment, the combination of water, glycerin, caprylyl/capryl glucoside pfaffia paniculata root extract, ptychopetalum olacoides extract and lilium candidum flower extract is in an amount of 1.8-2.2% by weight of the composition. In still another embodiment, the combination of water, medicago sativa extract, and hydrolyzed lupine protein is in an amount of 1.8-2.2% by weight of the composition, for example, 1.8, 1.9, 2.0, 2.1, and 2.2%. In another embodiment, the combination of sodium hyaluronate, water and phenoxyethanol is in an amount of 1.8-2.2% by weight of the composition, for example, 1.8, 1.9, 2.0, 2.1, and 2.2%.

A. Natural Products

i. Albizia julibrissin

Albizia julibrissin, also known as Silk Tree or Mimosa of Constantinople, is a deciduous tree of the Mimosaceae family. It is native to East Asia and South America. Albizia julibrissin is formulated in different known marketed cosmetic ingredients. For example, BEAUTIFEYE™ is a formulation of Albizia Julibrissin extract and darutoside in a glycerin excipient.

ii. Pfaffia paniculata

Pfaffia paniculata is a plant belonging to the botanical family, Amaranthaceae. This plant is of Amazonian origin and widely found in Brazil. A commercially available product comprising extracts of Pfaffia paniculata in combination with suitable carriers and other natural product extracts is BIOSKINUP CONTOR®.

iii. Ptychopetalum olacoides

Ptychopetalum olacoidesis is a plant belonging to the botanical family Olacaease. This plant is of South American origin. The bark and root of Ptychopetalum olacoides is harvested and used in herbal products. A commercially available product comprising extracts of Ptychopetalum olacoidein combination with suitable carriers and other natural product extracts is BIOSKINUP CONTOR®.

iv. Lilium candidum

Lilium candidum (White lily) is a member of the family Liliaceae with genus Lilium. Suitable extracts Lilium candidum may be derived from live or dried plant, small cuttings or other portions thereof. A commercially available product comprising extracts of Lilium candidum in combination with suitable carriers and other natural product extracts is BIOSKINUP CONTOR®.

v. Medicago sativa (Alfalfa)

Medicago sativa, also called lucerne, is a perennial flowering plant in the pea family Fabaceae. This plant is cultivated as a forage crop in many countries around the world. A commercially available product comprising extracts of Medicago sativa is EYEREGENEX®.

vi. Darutoside

Darutoside is a sugar derivative of darutigenol which has following chemical formula:

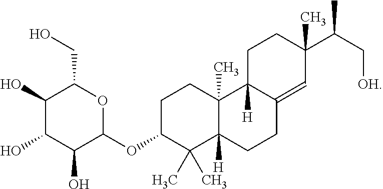

Darutoside may be obtained from a commercial supply source, chemical synthesis, enzymatic synthesis by one of the many methodologies of biotechnology, or by plant extraction from siegesbeckia orientalis.

B. Hyaluronic Acid

Hyaluronic acid is a naturally occurring, water soluble polysaccharide, specifically a glycosaminoglycan, which is a component of the extra-cellular matrix. This polysaccharide is widely distributed in human tissues. Hyaluronic acid has the ability to bind to large amounts of water, making it an excellent volumizer of soft tissues. Hyaluronic acid has a weight-average molecular weight ranging from 50 to 3,000 kDa, preferably ranging from 50 to 2500 kDa and preferentially ranging from 500 to 2,000 kDa. As illustrations of different hyaluronic acid fractions, reference is made to Stern, R. et al., “Hyaluronan fragments: An Information-Rich System”, European Journal of Cell Biology, 2006, 58, 699-715. This article provides biological activities of hyaluronic acid as a function of its molecular weight.

Salts of hyaluronic acid are pharmaceutically acceptable, preferably dermatologically acceptable. The salts of hyaluronic acid are selected from the hydrolyzed calcium hyaluronate, hydrolyzed sodium hyaluronate, calcium hyaluronate, potassium hyaluronate, sodium hyaluronate, sodium hyaluronate and sulfated mixtures. All such forms of hyaluronic acid are encompassed.

Hyaluronic acid may be provided by the company Hyactive under the trade name CPN (MW: 10 to 150 kDa), by the company Soliance under the trade name CRISTALHYAL® (MW: 1.1×10⁶), by the company Bioland under the name NUTRA® HA (MW: 820 kDa), by the company Bioland under the name NUTRA® HA (MW: 69 ka), by the company Bioland under the name OLIGO® HA (MW: 6100 Da) or by the company Vam Farma Cosmetica under the name D FACTOR® (MW: 380 Da), by the company LCA Pharmaceutical under the trade name HYALUDERM®, by the company Corneal under the trade name JUVELIFT CORNEAL®, by the company Q-Med under the trade name RESTYLANE® Touch Line, or by the company Revitacare under the trade name REVITACARE BIOREVITALIZATION®.

C. Dermatological Agents

The composition can also contain dermatological agents for improving the appearance and comfort of the skin. The dermatological agent can be an antioxidant. Examples of antioxidants include, but are not limited to, tocopheryls, retinoids, BHT, camellia sinensis leaf extract, carotenoids, resveratrol, triethyl citrate, arbutin, superoxide dismutase, zinc, sodium metabisulfite, lycopene, and ubiquinone or combinations thereof. Preferred antioxidants include retinol palmitate, panthenyl triacetate, and tocopheryl acetate. In preferred embodiments, the dermatological agents are a combination of tocopheryl acetate, adenosine and aminopropyl ascorbyl phosphate. In one embodiment, tocopheryl acetate, adenosine and aminopropyl ascorbyl phosphate are in an amount of up to about 10%, more preferably up to 5%, most preferably about 1.2% weight of the composition.

D. Excipients

The active agent composition can typically contain one or more cosmetically acceptable excipients. Cosmetically acceptable excipients include, but are not limited to, water, preservatives, chelating agents, sunscreen agents, vitamins, dyes, pigments proteins, amino acids, natural extracts such as plant extracts, humectants, fragrances, perfumes, oils, emollients, lubricants, butters, penetrants, thickeners, viscosity modifiers, polymers, resins, film formers, surfactants, detergents, emulsifiers, opacifying agents, volatiles, propellants, liquid vehicles, carriers, salts, pH adjusting agents (e.g., citric acid), neutralizing agents, buffers, absorbents, and combinations thereof.

i. Pigments

Pigments are used as coloring agents that provide color to cosmetic compositions. Pigments include, but are not limited to, titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, metal powders (e.g. aluminium powder and copper powder), cochineal carmine, organic pigments of azo dyes, anthraquinone dyes, indigoid dyes, xanthene dyes, pyrene dyes, quinoline dyes, triphenylmethane dyes and fluoran dyes. In one embodiment, the pigment is selected from the group consisting of titanium dioxide (CI177891), red iron oxide (CI177491), yellow iron oxides (CI177492), black iron oxide (CI177499), bismuth oxychloride, and combinations thereof.

The pigments can be included in a concentration ranging from 0.1 up to 20% of the total composition individually, depending on the desired resultant color of the composition. However, the pigments are preferably in a maximum concentration of about 20%, preferably, about 22%

Pigment particles, if used herein, can be surface modified with a hydrophobic coating such as, for example, triethoxycaprylsilane, methicone, or dimethincone, which is commonly known. In a preferred embodiment, the hydrophobic coating is triethoxycaprylsilane.

ii. Emollients

Emollients protect against wetness or irritation, softens, soothes, coats, lubricates, moisturizes, protects, and/or cleanses the skin. Suitable emollients for include, but are not limited to, a silicone compound (e.g., dimethicone, cyclomethicone, dimethicone copolyol or a mixture of cyclopentasiloxane and dimethicone/vinyldimethicone cross polymer, cyclopentasiloxane polysilicone), polyols such as sorbitol, glycerin, propylene glycol, ethylene glycol, polyethylene glycol, caprylyl glycol, polypropylene glycol, 1,3-butane diol, hexylene glycol, isoprene glycol, xylitol, ethylhexyl palmitate, a triglyceride such as caprylic/capric triglyceride and fatty acid ester such as cetearyl isononanoate or cetyl palmitate. More than one emollient may be included in the formulation. In an embodiment, the emollient is cyclopentasiloxane

iii. Emulsifiers

The compositions can contain one or more emulsifiers. Suitable emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, or polysorbate-80, or combinations thereof. More than one emulsifier may be included in the formulation. In preferred embodiments, the emulsifier is a combination of cetyl PEG, PPG-10, and 1-dimethicone, cetyl dimethicone and combinations thereof. In one embodiment, the cetyl PEG, PPG-10, and 1-dimethicone, cetyl dimethicone are in an amount of cetyl PEG, PPG-10, and 1-dimethicone and cetyl dimethicone are in an amount of up to about 10%, more preferably about 8.6% weight of the composition. In preferred embodiment, the compositions contain caprylyl/capryl glucoside.

iv. Thickeners

The composition can be thickened or structured with colloidal particles including, but not limited to, disteardimonium hectorite, kaolin, silica, and magnesium carbonate, viscous hydrocarbons, and combinations thereof. The concealer composition may be thickened with one or more wax(es) such as, for example, carnauba wax, candellila wax, beeswax, and polyethylene wax. This may help to provide proper spreading/deposition across a target skin surface and adequate stability/suspension of colorant particles in the dispersion over time.

v. Preservatives

One or more preservatives may be included in the composition to prevent microbial growth. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben), sodium benzoate, ethylenediamine-tetraacetic acid (EDTA), potassium sorbate, and/or grapefruit seed extract, or combinations thereof. More than one preservative may be included in the formulation. Other preservatives are known in the cosmetics industries and include salicylic acid, DMDM hydantoin, formaldahyde, chlorphenism, triclosan, imidazolidinyl urea, diazolidinyl urea, sorbic acid, methylisothiazolinone, sodium dehydroacetate, dehydroacetic acid, quaternium-15, stearalkonium chloride, zinc pyrithione, sodium metabisulfite, 2-bromo-2-nitropropane, chlorhexidine digluconate, polyaminopropyl biguanide, benzalkonium chloride, sodium sulfite, sodium salicylate, citric acid, neem oil, essential oils, lactic acid, and vitamin e (tocopherol).

An exemplary list of components that can be included in the disclosed compositions is shown in Table 1, below, including the most preferred concentrations and source of each component.

| | | | Batch |
|---|---|---|---|
| Ingredient | | | Weight |
| (Tradename) | INCI Designation | Supplier | (%) |
| ABIL ® EM 90 | Cetyl Peg/Ppg-10/1 | Evonik/UPI | 5.00 |
| | Dimethicone | | |
| ABIL ® Wax 9801 | Cetyl Dimethicone | Evonik/UPI | 3.60 |
| GRANSIL ® Gcm-5 | Cyclopentasiloxane | Grant | 11.50 |
| | Polysilicone-11 | | |
| BENTONE ® Gel | Caprylic/Capric Triglyceride | Elementis/Essential | 7.50 |
| Gtcc-V | Stearalkonium Hectorite | Ingredients | |
| | Propylene Carbonate | | |
| | Cyclopentasiloxane | | |
| XIAMETER ® Pmx- | Cyclopentasiloxane | Dow Corning | 7.53 |
| 0245 | | | |
| GRANSIL Eps ® | Polysilicone-11 Laureth-12 | Grant | 1.00 |
| Castor Wax Mp-70 | Hydrogenated Castor Oil | Vertellus/Hector | 2.00 |
| LIPOVOL ® Mos- | Tridecyl Stearate Tridecyl | Lipo | 5.00 |
| 130 | Trimellitate Dipentaerythrityl | | |
| | Hexacaprylate/Hexacaprate | | |
| ALCOLEC Bs ® | Lecithin | American | 0.50 |
| | | Lecithin | |
| Vitamin E Acetate - | Tocopheryl Acetate | BASF/DeWolf | 0.10 |
| BASF | | | |
| TINOGARD ® Tt | Pentaerythrityl Tetra-Di-T- | Ciba/BASF | 0.05 |
| | Butyl Hydroxyhydrocinnamate | | |
| UNIPURE ® White | titanium dioxide CI 77891, | Sensient Colours | 18.52 |
| LC 981 AS/Unipure | Triethoxycaprylylsilane | | |
| Titanium AS | | | |
| UNIPURE ® Red | Iron Oxides CI 77491, | Sensient Colours | 0.29 |
| LC 381 As | Triethoxycaprylylsilane | | |
| UNIPURE ® Yellow | Iron Oxides CI 77492, | Sensient Colours | 1.09 |
| LC 182 AS | Triethoxycaprylylsilane | | |
| UNIPURE ® Black | Iron Oxides CI 77499, | Sensient Colours | 0.10 |
| LC 989 AS | Triethoxycaprylylsilane | | |
| RONAFLAIR ® | Bismuth Oxychloride | Emd/Superior | 2.00 |
| Fines | | Material | |
| Deionized Water | Water (Aqua) | | 17.14 |
| KELTROL ® CG | Xanthan Gum | CP Kelco/Univar | 0.10 |
| Butylene Glycol - | Butylene Glycol | Celanese/UPI | 2.00 |
| UPI | | | |
| Adenosine #104750 | Adenosine | Premier | 0.02 |
| | | Specialties | |
| Versene Na EDTA | Disodium EDTA | Dow | 0.10 |
| USP | | Chemical/Univar | |
| Magnesium Sulfate, | Magnesium Sulfate | UPI | 0.10 |
| Anhydrous CAS | | | |
| 7487-88-9 | | | |
| K3 Vita C | Aminopropyl Ascorbyl | Ltrachem | 0.50 |
| | Phosphate | | |
| Natural Anhydrous | Caffeine | MMP | 0.25 |
| Caffeine USP | | | |
| Glycerin 99.7% | Glycerin | Acme-Hardesty | 1.00 |
| (Kosher) USP | | | |
| SYMDIOL ® 68 | 1,2-Hexanediol, Caprylyl | Symrise/Vigon | 1.00 |
| | Glycol | | |
| HYDROLITE ® 5 | Pentylene Glycol | Symrise/Vigon | 1.00 |
| EYE REGENER ® | Water Medicago Sativa | Silab | 2.00 |
| PX | (Alfalfa) Extract, Hydrolyzed | | |
| | Lupine Protein | | |
| HYANIFY | Water, Propanediol, Disodium | Lipotec | 1.00 |
| SOLUTION ® | Phosphate, Xantham Gum, | | |
| Bi030 | Saccharide Isomerate, Sodium | | |
| | Phosphate Glyceryl Caprylate | | |
| BIOSKINUP | Water, Glycerin, | Chemyunion/ | 2.00 |
| CONTOUR 3r ® | Caprylyl/Capryl Glucoside, | Taos | |
| | Pfaffia Paniculata Root Extract | | |
| | Ptychopetalum Olacoides | | |
| | Bark/Stem Extract, Lilium | | |
| | Candidum Flower Extract | | |
| HYASOL PF ® | Sodium Hyaluronate Water | Centerchem | 2.00 |
| | Phenoxyethanol | | |
| BEAUTIFEYE ® | Glycerin, Albizia Julibrissin | Croda/Sederma | 3.00 |
| | Bark Extract, Darutoside | | |
| Deionized Water | Water (Aqua) | | 1.00 |
| BASHYAL ® | Sodium Hyaluronate | Soliance | 0.01 |
| Powder | | | |

### III. Methods of Manufacture

A. Preparation of Natural Product Extracts

A typical process of preparing the crude natural product material for extraction is as follows:
1. The crude dry natural product materials are milled into fine powders using a milling device. Any grinding operation that achieves the respective particle size for extraction is acceptable. Suitable particle sizes are in the range of 100 microns to 1,000 microns. Milling is necessary to ensure that that crude, dried plant material is consistently-sized. Crude natural product extractability is dependent on the ratio of the exposed surface area of crude natural product powder to the mass of the hydroalcohol solvent. Other extraction solvents that can be used include, but are not limited to, ethanol, propanol, hot or cold water, diethyl ether, tetrahydrofuran, ethyl acetate, and combinations thereof. To eliminate crude natural product particle size as a process variable and since the various natural products have different water-holding capabilities (porosity/absorptivity), a singular particle size is preferred for process control. Depending on the specific type of crude natural product, milling produces a mix of coarse and fine dust particulates.
2. All milled crude natural product powders are mixed in a blender to provide a uniform particle size of the crude natural product powder prior to extraction. The particle size of the milled crude natural product powder is consistent following this step.
3. The crude natural product powder can be extracted with hydroalcohol solutions using a variety of methods. Two suitable methods for preparing the compositions described herein are described below.
   a) Soxhlet method: approximately 1-60 parts of milled crude natural product powder are added to 100-5000 parts (process and/or deionized or equivalent grade) water:alcohol in a Soxhlet Extractor and then decanted. The powder is generally extracted for a period of up to 48 hours.
   b) Ultrasonics method: a suitable alternate extraction process for preparing the water soluble extract includes the use of ultrasonic water extraction systems which can provide equivalent quality, depending on the natural product, with up to 94% faster process cycles. Suitable ultrasonic water extraction systems includes hydrolysis extracting reactors, fixed bed extracting reactors, desorption extraction columns, and countercurrent extractors.
4. The water-extracted natural product liquid is filtered (e.g., 5-100 micron filter cartridge, fine screen or cheesecloth) or centrifuged to remove coarse and/or insoluble particulates.
5. The filtered water-extracted natural product liquid is concentrated, depending on natural product ingredient, up to a 50% soluble solids level. In addition to concentration by evaporation, alternate suitable processes to achieve higher concentrations prior to final drying include freeze concentration, partial freeze drying, membrane separation, vacuum distillation and vacuum drying.
6. The concentrated natural product extract liquids are dried via commercial drying processes. Suitable dryers that can be used include fluidized bed, vacuum plate, spray, drum-type and flash dryers. Drying efficiency is controlled for water content (<10%) and free water considerations (<0.80) to achieve shelf-stability. The yield of soluble powder from the drying process is used as a key to optimize the natural product: water mass ratio for extraction.
7. The dried pure solid natural product extract powders are sized and packaged for shipment. Desiccating materials such as a silica gel or other suitable FDA-approved, drying agents can be used to control relative humidity and to improve shelf-life.
8. The dried pure solid natural product extract powder is now ready for reconstitution into cosmetic care products.

B. Manufacture of Formulations

The composition can be in various galenical forms conventionally used for topical applications and in particular in the form of dispersions of the lotion or serum type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or wax/aqueous phase dispersions. These compositions are prepared according to the well-known methods to those skilled in the art.

In addition, the compositions may be more or less fluid and may have the appearance of a gel, a white or colored cream, an ointment, a milk, a lotion, a serum, a paste or a mousse.

In one preferred embodiment, the composition is in the form of an oil-in-water emulsion. The composition may also be in the form of an emulsified gel. In another embodiment, the composition is in the form of an O/W emulsion. In yet another embodiment, the composition is in the form of a W/O emulsion. In preferred embodiments, the composition is formulated as a solution.

The composition preferably has a skin-friendly pH which generally ranges from 4 to 8 and preferably from 4.5 to 6.5.

### IV. Methods of Use

Methods for concealing undesirable blemishes, discolorations and/or other perceived cosmetic defects on skin are disclosed herein. In particular, the compositions lighten the skin by whitening, and/or evening of the skin tone, skin color, and/or shade of skin, and/or to the reduction in shallowness, and/or to the lightening and/or fading of hyperpigmented marks and/or lesions including, but not limited to, pigmented spots, melanin spots, age spots, sun spots, senile lentigos, freckles, lentigos simplex, pigmented solar keratosis, seborrhoeic keratosis, melasma, acne marks, post-inflammatory hyperpigmentation, lentigines, ephelides, and combinations of two or more thereof.

Concealers can be applied to relatively small areas of the face (e.g., under-eye area) but are not intended to be applied to the entire face. The compositions also find their use in a very large number of cosmetic treatments for the skin, including the scalp, and the mucous membranes (lips). The compositions can be used for reducing the visible or tactile irregularities of the surface of the skin, in particular for reducing wrinkles and fine lines and/or blemishes on the skin and/or smoothing and/or firming the skin and/or unifying the complexion.

### EXAMPLES

### Example 1

### Manufacture of Composition 1

### Materials:

TABLE 2
Phase A Ingredients

| | | | |
|---|---|---|---|
| | | | Batch |
| Ingredient | | | Weight |
| (Tradename) | INCI Designation | Supplier | (%) |

Phase A

| | | | |
|---|---|---|---|
| ABIL ® EM 90 | Cetyl Peg/Ppg-10/1 | Evonik/UPI | 5.00 |
| | Dimethicone | | |
| ABIL ® Wax 9801 | Cetyl Dimethicone | Evonik/UPI | 3.60 |
| GRANSIL ® Gcm-5 | Cyclopentasiloxane | Grant | 11.50 |
| | Polysilicone-11 | | |
| BENTONE ®Gel | Caprylic/Capric Triglyceride | Elementis/Essential | 7.50 |
| Gtcc-V | Stearalkonium Hectorite | Ingredients | |
| | Propylene Carbonate | | |
| | Cyclopentasiloxane | | |
| XIAMETER ® Pmx- | Cyclopentasiloxane | Dow Corning | 7.53 |
| 0245 | | | |
| GRANSIL Eps ® | Polysilicone-11 Laureth-12 | Grant | 1.00 |
| Castor Wax Mp-70 | Hydrogenated Castor Oil | Vertellus/Hector | 2.00 |
| LIPOVOL ® Mos- | Tridecyl Stearate Tridecyl | Lipo | 5.00 |
| 130 | Trimellitate Dipentaerythrityl | | |
| | Hexacaprylate/Hexacaprate | | |
| ALCOLEC Bs ® | Lecithin | American | 0.50 |
| | | Lecithin | |
| Vitamin E Acetate - | Tocopheryl Acetate | BASF/DeWolf | 0.10 |
| BASF | | | |
| TINOGARD ® Tt | Pentaerythrityl Tetra-Di-T- | Ciba/BASF | 0.05 |
| | Butyl Hydroxyhydrocinnamate | | |

Phase A ingredients were weighed and placed in the reaction vessel. The mixture was heated to 80° C. and until uniform with homomixing.

TABLE 3
Phase B Ingredients

| | | | |
|---|---|---|---|
| | | | Batch |
| Ingredient | | | Weight |
| (Tradename) | INCI Designation | Supplier | (%) |

Phase B

| | | | |
|---|---|---|---|
| UNIPURE ® White | Iron Oxides CI 77891, | Sensient | 18.52 |
| LC 981 AS/Unipure | Triethoxycaprylylsilane | Colours | |
| Titanium AS | | | |
| UNIPURE ® Red | Iron Oxides CI 77491, | Sensient | 0.29 |
| LC 381 As | Triethoxycaprylylsilane | Colours | |
| UNIPURE ® Yellow | Iron Oxides CI 77492, | Sensient | 1.09 |
| LC 182 AS | Triethoxycaprylylsilane | Colours | |
| UNIPURE ® Black | Iron Oxides CI 77499, | Sensient | 0.10 |
| LC 989 AS | Triethoxycaprylylsilane | Colours | |
| RONAFLAIR ® | Bismuth Oxychloride | Emd/ | 2.00 |
| Fines | | Superior | |
| | | Material | |

Each Phase B ingredient was pulverized and added to the reaction vessel containing Phase A.

TABLE 4
Phase C and Phase d Ingredients

| | | | |
|---|---|---|---|
| | | | Batch |
| Ingredient | | | Weight |
| (Tradename) | INCI Designation | Supplier | (%) |

Phase C

| | | | |
|---|---|---|---|
| Deionized Water | Water (Aqua) | | 17.14 |
| KELTROL ® CG | Xanthan Gum | CP Kelco/Univar | 0.10 |
| Butylene Glycol - | Butylene Glycol | Celanese/UPI | 2.00 |
| UPI | | | |

Phase D

| | | | |
|---|---|---|---|
| Adenosine #104750 | Adenosine | Premier | 0.02 |
| | | Specialties | |
| Versene Na EDTA | Disodium EDTA | Dow | 0.10 |
| USP | | Chemical/Univar | |
| Magnesium Sulfate, | Magnesium Sulfate | UPI | 0.10 |
| Anhydrous CAS | | | |
| 7487-88-9 | | | |
| K3 Vita C | Aminopropyl Ascorbyl | Ltrachem | 0.50 |
| | Phosphate | | |
| Natural Anhydrous | Caffeine | MMP | 0.25 |
| Caffeine USP | | | |
| Glycerin 99.7% | Glycerin | Acme-Hardesty | 1.00 |
| (Kosher) USP | | | |
| SYMDIOL ® 68 | 1,2-Hexanediol, | Symrise/Vigon | 1.00 |
| | Caprylyl Glycol | | |
| HYDROLITE ® 5 | Pentylene Glycol | Symrise/Vigon | 1.00 |

Phase C ingredients were mixed for 30 minutes and heated to 75° C. Phase D ingredients were added one at a time to Phase C. This mixture containing Phase C and D ingredients was added to the reaction vessel containing Phase A and B. The reaction vessel was switched to sweep.

TABLE 5
Phase E Ingredients

| | | | |
|---|---|---|---|
| Ingredient | | | Batch Weight |
| (Tradename) | INCI Designation | Supplier | (%) |

Phase E

| | | | |
|---|---|---|---|
| EYE REGENER ® | Water Medicago Sativa | Silab | 2.00 |
| PX | (Alfalfa) Extract, Hydrolyzed | | |
| | Lupine Protein | | |
| HYANIFY | Water, Propanediol, Disodium | Lipotec | 1.00 |
| SOLUTION ® | Phosphate, Xantham Gum, | | |
| Bi030 | Saccharide Isomerate, Sodium | | |
| | Phosphate Glyceryl Caprylate | | |
| BIOSKINUP | Water, Glycerin, | Chemyunion/Taos | 2.00 |
| CONTOUR 3r ® | Caprylyl/Capryl Glucoside, | | |
| | Pfaffia Paniculata Root Extract | | |
| | Ptychopetalum Olacoides | | |
| | Bark/Stem Extract, Lilium | | |
| | Candidum Flower Extract | | |
| HYASOL PF ® | Sodium Hyaluronate Water | Centerchem | 2.00 |
| | Phenoxyethanol | | |
| BEAUTIFEYE ® | Glycerin, Albizia Julibrissin | Croda/Sederma | 3.00 |
| | Bark Extract, Darutoside | | |

Phase F

| | | | |
|---|---|---|---|
| Deionized Water | Water (Aqua) | | 1.00 |
| BASHYAL ® | Sodium Hyaluronate | Soliance | 0.01 |
| Powder | | | |

Phase E ingredients were premixed. At 40° C., Phase E was added to the reaction vessel containing Phases A, B, C, and D to afford the formulation of Composition 1.

### Example 2

### Clinical Trial Study

Objective

The purpose of this study was to evaluate the performance of a topically-applied skin lightening product (Composition 1) when tested over a 56 day period. The quantification of skin color changes associated with the use of the test material was evaluated via MINOLTA® CHROMAMETER® Color Computer System on a group of 30 panelists.

Standards for Inclusion

Females were between the ages of 35 and 65. The subjects exhibited the visible hyperpigmented areas under eyes as determined by the Trained Evaluator. Individuals completed a preliminary medical history and screening document.

Panel Demographics

The number of subjects enrolled in the study was thirty and thirty subjects completed the study. The race of the subjects was the following: Caucasian (26), Asian (2) and Hispanic (2).

Clinical Procedure

In order to pre-condition the test sites and keep all topical treatments consistent during the study, the panelists were required to abstain from using any self-tanning, anti-aging, skin lightening and moisturizing products, including lotions, creams, gels and nutritional supplements, for a period of at least 72 hours prior to study commencement.

On the day of the test, panelists reported to the test facility with their face area devoid of topical treatments and were examined by a trained technician. Upon arrival, panelists were allowed to equilibrate to the ambient environment for 30 minutes prior to measurement. Biophysical measurements were collected at baseline and again after 28 and 56 days of daily use of the test product. In addition, all subjects completed a self-assessment questionnaire addressing consumer perception immediately after product application and again after 28 and 56 days of daily use of the test treatment.

All participants were instructed to use the test material as a part of their daily routine according to the following sponsor-supplied use instructions:
The product comes in ten shades and it is to be used under your eye area one time per day in the morning.
To use the product dispense a small drop about a half the size of a pea onto your hand or finger.
To apply the product start at the inner corner of your eye and blend about ¾ of the way until your dark circle is covered. Repeat the same procedure on the other eye.
If you need more coverage the product layers and builds very well so you can add on top of what you have just applied.
Please do not use any other products around the eye when using this product.

The following distinct noninvasive method was employed to establish evaluation parameters:

Skin Color Brightness! Lightness (L*)—MINOLTA® CHROMAMETER®.

The MINOLTA® CR-200 CHROMAMETER® detects subtle changes in color by a three dimensional profile of hue, value and chroma. These characteristics are then translated into color coordinates (a*, b* and L*) whose spacing is considered to correlate with the color changes perceived by the human eye. Any increase in the L* coordinate indicates lightening of the color. Any diminution of the L* coordinate is indicative of the darkening of color.

| | L* Coordinate |
|---|---|
| Increase | Lightening |
| Decrease | Darkening |

The data on each of the 30 subjects are provided in Table 5. The data reflects changes in skin color where test site Baseline readings are considered 0% and the lightest (least dark—clear skin) skin color for each panelist is considered 100%. Clear skin is defined as a natural, untanned skin tone/color for each individual. Table 6 describes the efficacy of the composition at various time points.

TABLE 6
Skin Color Brightness/Lightness Data [L*]

| Baseline | Day 28/% Diff. | | Day 56/% Diff. | | Clear Skin |
|---|---|---|---|---|---|
| 61.40 | 62.37 | 40.28% | 62.23 | 34.72% | 63.80 |
| 63.07 | 63.93 | 22.81% | 63.40 | 8.77% | 66.87 |
| 59.50 | 60.20 | 12.57% | 61.30 | 32.34% | 65.07 |
| 57.70 | 58.80 | 16.75% | 60.20 | 38.07% | 64.27 |
| 57.43 | 60.63 | 40.51% | 58.23 | 10.13% | 65.33 |
| 49.27 | 50.33 | 8.67% | 53.60 | 35.23% | 61.57 |
| 59.57 | 60.70 | 39.08% | 60.93 | 47.13% | 62.47 |
| 58.13 | 57.22 | −11.20% | 59.27 | 13.93% | 66.27 |
| 56.13 | 55.90 | −8.24% | 56.23 | 3.53% | 58.97 |
| 58.03 | 59.67 | 22.37% | 60.53 | 34.25% | 65.33 |
| 57.60 | 57.57 | −0.46% | 57.60 | 0.00% | 64.80 |
| 56.33 | 57.63 | 21.31% | 57.47 | 18.58% | 62.43 |
| 62.87 | 63.50 | 19.39% | 64.13 | 38.78% | 66.13 |
| 49.13 | 54.67 | 49.11% | 54.80 | 50.30% | 60.40 |
| 58.97 | 60.90 | 20.14% | 61.00 | 21.18% | 68.57 |
| 57.70 | 58.73 | 19.87% | 60.80 | 59.62% | 62.90 |
| 51.90 | 55.60 | 45.12% | 55.00 | 37.80% | 60.10 |
| 61.00 | 61.57 | 22.97% | 62.63 | 66.22% | 63.47 |
| 54.03 | 55.90 | 27.05% | 57.27 | 46.86% | 60.93 |
| 56.63 | 56.90 | 3.28% | 57.23 | 7.38% | 64.77 |
| 57.27 | 57.77 | 5.79% | 57.87 | 6.95% | 65.90 |
| 51.23 | 55.03 | 57.29% | 56.00 | 71.86% | 57.87 |
| 59.80 | 59.37 | −6.28% | 60.67 | 12.56% | 66.70 |
| 56.57 | 58.43 | 30.94% | 59.50 | 48.62% | 62.60 |
| 52.47 | 54.43 | 30.41% | 53.70 | 19.07% | 58.93 |
| 59.80 | 60.50 | 16.15% | 60.43 | 14.54% | 64.13 |
| 54.70 | 56.60 | 19.72% | 57.00 | 23.88% | 64.33 |
| 62.47 | 62.17 | −28.12% | 62.40 | −6.25% | 63.53 |
| 64.77 | 65.07 | 12.50% | 64.80 | 1.39% | 67.17 |
| 60.30 | 60.50 | 4.96% | 60.30 | 0.00% | 64.33 |

TABLE 7
Skin Color Brightness/Lightness Data [L*]

Study Time Point:

| | Day 28 | Day 56 |
|---|---|---|
| % Difference: | 19.99%* | 27.56%* |
| Max % Improvement: | 57.29% | −71.86% |

*Statistically Significant

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I/we claim:

1. A topical cosmetic composition comprising:
(a) a combination of glycerin, albizia julibrissin extract, and darutoside;
(b) a combination of water, glycerin, caprylyl/capryl glucoside pfaffia paniculata root extract, ptychopetalum olacoides extract and lilium candidum flower extract;
(c) a combination of water, medicago sativa extract, and hydrolyzed lupine protein; and
(d) a combination of sodium hyaluronate, water and phenoxyethanol,
In effective amounts to lighten the skin.

2. The composition of claim 1, wherein the combination of (a) is in an amount of between about 2.8 and about 3.2% by weight of the composition.

3. The composition of claim 2, wherein the combination of (b) is in an amount of between about 1.8 and about 2.2% by weight of the composition.

4. The composition of claim 3, wherein the combination of (c) is in an amount of between about 1.8 and about 2.2% by weight of the composition.

5. The composition of claim 4, wherein the combination of (d) is in an amount of between about 1.8 and about 2.2% by weight of the composition.

6. The composition of claim 1 further comprising one or more pigments.

7. The composition of claim 1 further comprising one or more emulsifiers.

8. The composition of claim 1 further comprising one or more dermatological agents.

9. The composition of claim 1 further comprising one or more emollients.

10. The composition of claim 1 further comprising one or more preservatives.

11. The composition of claim 6, wherein the pigment is selected from the group consisting of iron oxide CI177891, iron oxide CI177491, iron oxide CI177492, iron oxide CI177499, and bismuth oxychloride, and combinations thereof.

12. The composition of claim 11 further comprising triethoxycaprylylsilane.

13. The composition of claim 12, wherein the pigments and triethoxycaprylylsilane are in an amount of between about 18 and about 22% weight of the composition.

14. The composition of claim 7, wherein the emulsifier is selected from the group consisting of cetyl PEG, PPG-10, and 1-dimethicone, cetyl dimethicone and combinations thereof.

15. The composition of claim 14, wherein cetyl PEG, PPG-10, and 1-dimethicone and cetyl dimethicone are in an amount of up to about 10%, more preferably about 8.6% weight of the composition.

16. The composition of claim 8, wherein the dermatologic agent is selected from the group consisting of tocopheryl acetate, adenosine and aminopropyl ascorbyl phosphate.

17. The composition of claim 16, wherein tocopheryl acetate, adenosine and aminopropyl ascorbyl phosphate are in an amount of up to about 10%, more preferably up to 5%, most preferably about 1.2% weight of the composition.

18. The composition of claim 9, wherein the emollient is cyclopentasiloxane

19. The composition of claim 10, wherein the preservative is phenoxyethanal or sodium EDTA.

20. The topical cosmetic composition of claim 1 formulated as a solution.

21. The topical composition of claim 1 formulated as a concealor.

22. A method for lightening the skin comprising administering the composition of claim 1.
